(19) United States
(12) Patent Application Publication
(10) Pub. No.: US 2026/0199676 A1
(43) Pub. Date: Jul. 16, 2026

(54) APPARATUS AND METHOD FOR RELIEVING SYMPTOMS OF ENDOMETRIOSIS

(71) Applicant: Z.A. ARGO LTD., Tiberias (IL)

(72) Inventor: Zvi NACHUM, Tiberias (IL)

(21) Appl. No.: 19/560,270

(22) Filed: Mar. 9, 2026

Related U.S. Application Data
(63) Continuation of application No. PCT/IB2024/058978, filed on Sep. 16, 2024.

(30) Foreign Application Priority Data
Sep. 18, 2023 (GB) 2314186.4

(51) Int. Cl. A61N 1/36; A61N 1/04

(52) U.S. Cl. CPC A61N 1/36021; A61N 1/0404; A61N 1/36007; A61N 1/3603

(57) ABSTRACT

An apparatus for relieving symptoms of endometriosis comprises an electrical stimulator comprising a plurality of electrodes configured to be placed in spaced-apart contact with skin of a subject. The apparatus comprises electronic circuitry providing, for the duration of a treatment session lasting at least two minutes, an electrical signal for application to the subject through the plurality of electrodes, the electrical signal comprising a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second.

### CROSS-REFERENCE TO RELATED APPLICATIONS

This application is a continuation of PCT/IB2024/058978 (filed on 16 Sep. 2024) which is incorporated herein by reference in its entirety. PCT/IB2024/058978 claims priority to U.K Patent Application GB2314186.4, filed Sep. 18, 2023, entitled “Apparatus and method for relieving symptoms of endometriosis”, which is incorporated herein by reference in its entirety.

### FIELD OF THE INVENTION

The present invention relates to non-invasive and nonhormonal treatments for relieving one or more symptoms of endometriosis, and in particular to treatments including transcutaneous electrical stimulation.

### BACKGROUND

Endometriosis is a nonmalignant disorder in which functioning endometrial tissue is present outside the uterine cavity. Endometriotic tissues contain estrogen and progestogen receptors that enable them to grow and differentiate in response to the changes in hormonal levels during the menstrual cycle. Clinical manifestations of endometriosis can include pelvic pain, pelvic mass, alteration of menses, and infertility. Endometriotic implants on the ovary or adnexal structures can form an endometrioma (a cystic mass localized to an ovary) or adnexal adhesions. Endometriosis is reportedly found in 10-15% of women between the ages of 25 and 44 who are actively menstruating, and in 25-50% of infertile women.

Treatments for endometriosis include medical suppression of ovarian function to arrest the growth and activity of endometrial implants, conservative surgical resection of as much endometriotic tissue as possible, a combination of these two treatments, and total hysterectomy, usually with removal of the ovaries and Fallopian tubes. Treatments additional include medical therapies involving estrogen suppression.

The application of transcutaneous electrical pulses has been previously disclosed to be effective in relieving pain in certain circumstances. Existing approaches have not proven adequate for relieving the pain of secondary dysmenorrhea nor for improving organic removal of endometrial tissue.

### SUMMARY

Embodiments of the present invention relate to apparatuses and methods for providing treatments for the relief of symptoms of endometriosis using transcutaneous electrical pulses.

According to embodiments, an apparatus for relieving symptoms of endometriosis comprises: (a) an electrical stimulator comprising a plurality of electrodes configured to be placed in spaced-apart contact with skin of a subject; and (b) electronic circuitry configured to provide, when activated for the duration of a treatment session lasting at least two minutes, an electrical signal for application to the subject through the plurality of electrodes, the electrical signal comprising a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of a positive-voltage pulse and a negative-voltage pulse. In some embodiments, each respective progression has a duration of at least 3 seconds and at most 20 seconds.

In some embodiments, a method of relieving one of more symptoms of endometriosis can comprise: (a) providing the apparatus of any one of the embodiments disclosed herein; (b) placing the plurality of electrodes in spaced-apart contact with skin of the subject; and (c) providing the electrical signal for a duration of a treatment session lasting multiple minutes, wherein the electrical signal comprises a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of a positive-voltage pulse and a negative-voltage pulse, wherein each respective progression has a duration of at least 3 seconds and at most 20 seconds. In some embodiments of the method, the providing of the electrical signal can be effective, during the treatment session, (i) to relieve a pain of secondary dysmenorrhea and (ii) to promote, increase an amplitude of, and/or increase a frequency of, uterine contractions.

A method is disclosed, according to embodiments of the invention, for relieving one or more symptoms of endometriosis. The method comprises: (a) providing an apparatus comprising: (i) an electrical stimulator comprising a plurality of electrodes, and (ii). electronic circuitry configured to provide an electrical signal to a subject through the plurality of electrodes. The method also comprises (b) placing the plurality of electrodes in spaced-apart contact with skin of the subject; and (c) providing the electrical signal for a duration of a treatment session lasting multiple minutes, wherein the electrical signal comprises a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of a positive-voltage pulse and a negative-voltage pulse. In some embodiments, each respective progression has a duration of at least 3 seconds and at most 20 seconds.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIG. 1 is simplified block diagram of an apparatus for relieving one or more symptoms of endometriosis, according to embodiments of the present invention.

FIG. 2 is a schematic illustration of an apparatus for relieving one or more symptoms of endometriosis, according to embodiments of the present invention.

FIG. 3 is a schematic electrical diagram showing selected elements of an apparatus for relieving one or more symptoms of endometriosis, according to embodiments of the present invention.

FIGS. 4A and 4B are schematic illustrations of exemplary pulsing cycles, according to embodiments of the present invention.

FIG. 5 shows a schematic graph illustrating an exemplary progression of pulsing frequencies during a single sequence, according to embodiments of the present invention.

FIG. 6 shows a schematic graph illustration an exemplary progression of pulsing frequencies followed by an exemplary degression of pulsing frequencies during a single sequence, according to embodiments of the present invention.

FIGS. 7A, 7B and 7C are schematic illustrations of the pulsing cycle, showing the initial pulse width and amplitude of FIG. 4B in FIG. 7A, an expanded pulse width in FIG. 7B, and a reduced pulse amplitude in FIG. 7C, according to embodiments of the present invention.

FIGS. 8A and 8B show schematic graphs illustrating consecutive sequences of progressions, respectively without and with an intervening user-selected rest period, according to embodiments of the present invention.

FIGS. 9A and 9B show schematic graphs illustrating consecutive sequences of progressions followed by degressions, respectively without and with an intervening an intervening user-selected rest period between sequences, according to embodiments of the present invention.

FIGS. 10A, 10B and 10C show schematic graphs illustrating the exemplary progression of FIG. 6, where in FIG. 10A the progression is applied according to an initial progression duration, in FIG. 10B the progression is applied according to a user-shortened progression duration, and in FIG. 10C the progression is applied according to a user-lengthened progression duration, according to embodiments of the present invention.

FIGS. 11A, 11B and 11C show schematic graphs illustrating the exemplary progression of FIG. 6, where in FIG. 11A the frequency is monotonically increased in an initial number of steps, in FIG. 11B the frequency is monotonically increased in a user-reduced number of steps, and in FIG. 11C the frequency is monotonically increased in a user-increased number of steps, according to embodiments of the present invention.

FIGS. 12, 13A, 13B, 13C, 13D, 13E and 13F show flowcharts of methods and method steps for relieving one of more symptoms of endometriosis, according to embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Some embodiments of the invention relate to apparatuses and methods for applying transcutaneous electrical pulses to a user's torso for relief of symptoms of endometriosis. Where the term ‘relief of symptoms’ (or similar) is used herein, the term should be understood to include at least partial relief, and at least temporary relief, as well as complete relief and/or long-term relief. Some of the symptoms believed to be relievable by the apparatus and/or methods of the instant embodiments include painful menses, considered secondary dysmenorrhea, and insufficient removal of endometrial tissue during menses. The inventors are of the opinion that inducing uterine contractions, including increasing the frequency and/or intensity of uterine contractions, can contribute to more effective removal or expulsion of endometrial tissue during menses. Thus, the inducing of uterine contractions is related to in the disclosure as relieving one or more symptoms of endometriosis, alongside relief of pain. Without being bound by theory, the uterine contractions can be caused by peristaltic action of selected muscle groups engendered by chemical production, e.g., of acetylcholine, in response to cyclical electrical stimulation, e.g., in electrical-pulse progressions of monotonically increasing frequency as disclosed herein. The contractions induced or affected by the cyclical electrical stimulation can then replace or supplement the ‘natural’ contractions of menses to aid in removal of endometrial tissue.

The inventors have found that relief of the one or more symptoms of endometriosis can be achieved by applying the electrical pulses to the user's torso in successive sequences comprising respective progressions of pulsing cycles. Each pulsing cycle includes at least one of a positive-voltage pulse and a negative-voltage pulse, i.e., at least one positive-voltage pulse and/or at least one negative-voltage pulse. In a first non-limiting example, a pulsing cycle includes exactly one positive-voltage pulse and no negative-voltage pulses. In a second non-limiting example, a pulsing cycle includes exactly one positive-voltage pulse and exactly one negative-voltage pulse of substantially equal intensity, width (duration) and/or electrical energy as the preceding positive-voltage pulse. Alternating positive-voltage pulses and negative-voltage pulses is known to avoid polarization.

A ‘progression’ comprises pulsing cycles increasing in frequency throughout the progression, e.g., monotonically increasing in frequency. In an illustrative example, a progression begins with pulsing cycles applied at a frequency of 50 pulsing cycles per second, and ends with pulsing cycles applied at a frequency of 150 pulsing cycles per second. In other examples, the starting (minimum) frequency for a progression may be as low as 30 pulsing cycles per second and as high as 60 pulsing cycles per second. The ending (maximum) frequency for a progression may be as low as 100 pulsing cycles per second and as high as 200 pulsing cycles per second. In embodiments, there is a frequency increase, from the starting or minimum frequency to the ending or maximum frequency, of at least 40 pulsing cycles per second during a progression, or at least 45 pulsing cycles per second, or at least 50 pulsing cycles per second, or at least 60 pulsing cycles per second, or at least 70 pulsing cycles per second, or at least 80 pulsing cycles per second, or at least 90 pulsing cycles per second, or at least 100 pulsing cycles per second. In embodiments, a user may adjust one or more of the parameters of the progression for comfort and/or efficacy of inducing or regulating the frequency and/or intensity of contractions.

An exemplary progression may last as little as 3 seconds or as long as 20 seconds. The progression may include at least 20 steps (frequency increases) or as many as 200 steps. A minimum step is an increase of at least 1 pulsing cycle per second; a step may include an increase of as many as 10 pulsing cycles per second. In embodiments, a treatment session can include at least 10, or at least 20, or at least 50 successive sequences. An apparatus according to embodiments can be programmed to provide the progressions for a selected duration of a treatment session, and/or can be pre-set to a default duration.

In some implementations, each sequence comprises only a progression such as disclosed herein, and then the sequence is repeated. In some implementations, a rest period of any desired duration, i.e., without application of electrical pulses, can separate progressions. In some implementations, each progression is followed by a degression comprising pulsing cycles monotonically decreasing in frequency throughout the degression. The degression can be a mirror image of the progression, starting at a maximum pulsing cycle frequency equal to, or within ±5% of, or within ±10% of, or within ±15% of the maximum pulsing cycle frequency of the progression, and decreasing to a minimum pulsing cycle frequency equal to, or within ±5% of, or within ±10% of, or within ±15% of the minimum pulsing cycle frequency of the progression. The degression can have the same duration of the progression, or have a duration that is within ±15% of the duration of the progression. In some embodiments, the degression does not contribute to the therapeutic nature of the treatment but rather is implemented to allow the user to feel greater comfort (or less discomfort) and have a smoother transition between progressions. In some implementations, the user can opt for a rest period, i.e., without application of electrical pulses between sequences, i.e., after each degression and before the beginning of the progression of the next sequence, or between successive progressions if no degressions are included in the sequences.

Treatment sessions including applications of successive sequences of pulse cycles are preferably of a duration long enough to elicit some beneficial results to the user, and thus are not insubstantial. In embodiments, treatment sessions last multiple minutes, e.g., at least two minutes, or at least 3 minutes, or at least 4 minutes, or at least 5 minutes, or at least 6 minutes, or at least 7 minutes, or at least 8 minutes, or at least 9 minutes, or at least 10 minutes, or longer.

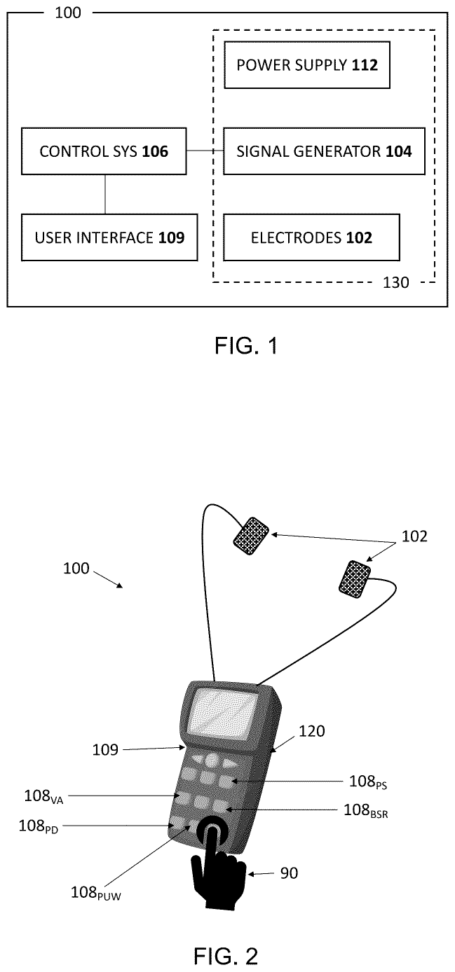

Referring now to the figures and in particular to FIG. 1, a simplified block diagram is shown of an apparatus 100 according to embodiments. The apparatus 100 is an apparatus for relieving one or more symptoms of endometriosis, and includes an electrical stimulator 130 for generating and applying transcutaneous electrical pulses to a user, electronic circuitry comprising a control system 106 for controlling the electrical stimulator 130, and a user interface 109 for communicating with a user or caregiver, including receiving user inputs related to the operation of the apparatus 100 as well as providing information to the user or caregiver. The electrical stimulator 130 includes a power supply 112, a signal generator 104 for generating an electrical signal comprising a plurality of successive sequences of electrical pulses according to embodiments, and at least two electrodes 102. The electrodes 102 are provided with wires allowing the electrodes 102 to be placed in spaced-apart contact with skin of a subject. The control system 106 is configured, e.g., programmed, to cause the electrical stimulator 130 to provide, as described hereinabove, an electrical signal for application to a subject through the plurality of electrodes 102, the electrical signal comprising a plurality of successive sequences of pulsing cycles pulses, each sequence comprising at least respective progressions.

A schematic illustration of an apparatus 100 is shown in FIG. 2, as a non-limiting example, in a hand-held configuration. In other examples, not illustrated, the apparatus can have a tabletop or cart-top configuration or can be installed in an instrument cabinet. In the example of FIG. 2, the user interface 109 includes a number of function-specific input mechanisms 108 (e.g., buttons, switches, dials, whether physical/mechanical or on-screen) for receiving user inputs. For example:

Input mechanism 108_{VA} is for receiving a voltage adjustment input from a user 90.

Input mechanism 108_{PD} is for receiving a progression-duration adjustment input from a user 90.

Input mechanism 108_{PUW} is for receiving a pulse-width adjustment input from a user 90.

Input mechanism 108_{PS} is for receiving an input from a user 90 to adjust the number of steps in the progression.

Input mechanism 108_{BSR} is for receiving an input from a user 90 to add, delete or adjust a between-sequences rest period.

In the non-limiting example of FIG. 2, exactly two electrodes 102 are shown. In some embodiments, the apparatus 100 includes a port (not shown) for attaching the electrodes 102.

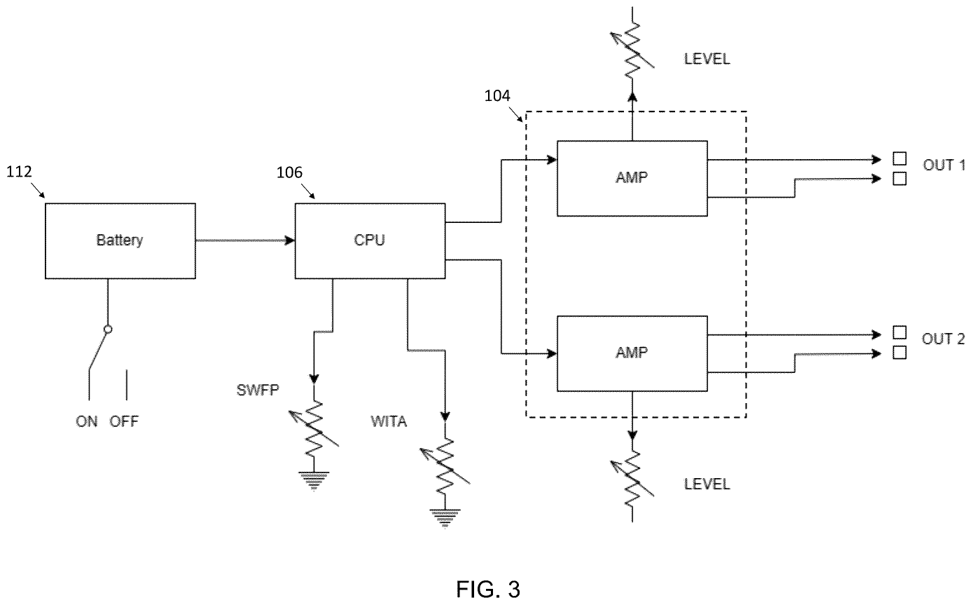

We now to refer to FIG. 3, which shows a simplified line diagram of an exemplary apparatus 100 according to embodiments. The power source 112 is represented in FIG. 3, by a battery, e.g., a 3V primary or rechargeable battery, although any source of electricity can be used. The control system 106 is represented by a CPU and representative components, e.g., variable resistors, for adjusting any of the user-adjustable parameters disclosed above, including any or all of voltage adjustment, progression-duration adjustment, pulse-width adjustment, adjustment of the number of steps in a progression, and addition or adjustment of a between-sequences rest period. In the non-limiting example of FIG. 3, a voltage amplifier is provided for each output channel; in some designs, a separate CPU can be provided for each channel.

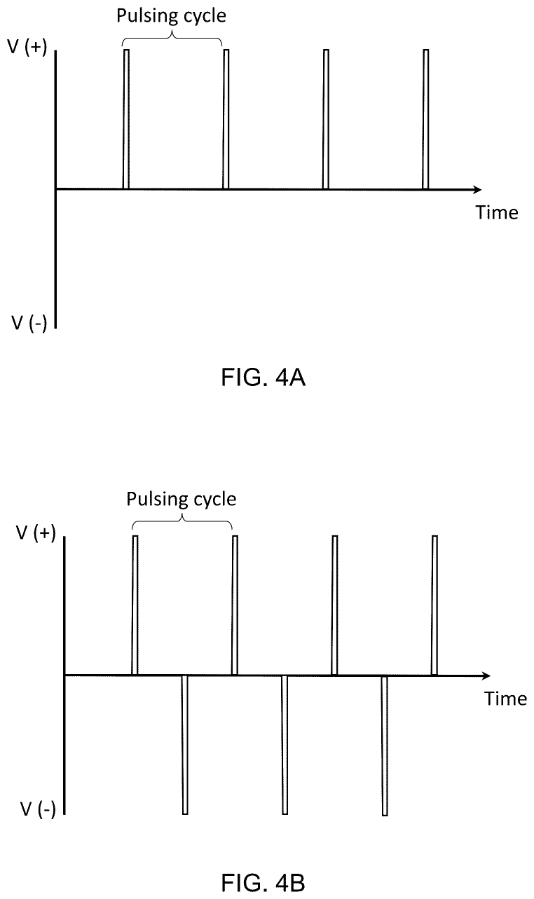

FIGS. 4A and 4B illustrate two examples as described hereinabove of pulse-cycle sequences. FIG. 4A illustrates, in a schematic graph of voltage vs. time, pulse cycles comprising only positive-voltage pulses. In contrast, FIG. 4B illustrates, in a similar schematic graph of voltage vs. time, pulse cycles comprising positive-voltage pulses followed by negative-voltage pulses. It can be desirable for the negative-voltage pulses and positive-voltage pulses to be evenly spaced out, e.g., with the negative-voltage pulses times to be approximately halfway between consecutive positive-voltage pulses. FIGS. 4A and 4B are not drawn to scale, and both pulse widths (pulse durations) and lengths of the pulsing cycles are variable. In an example, pulse width is between 100 and 300 microseconds, and the pulsing-cycle duration is between 5 and 33 milliseconds (corresponding to a maximum frequency range of 30 to 200 pulsing cycles per second).

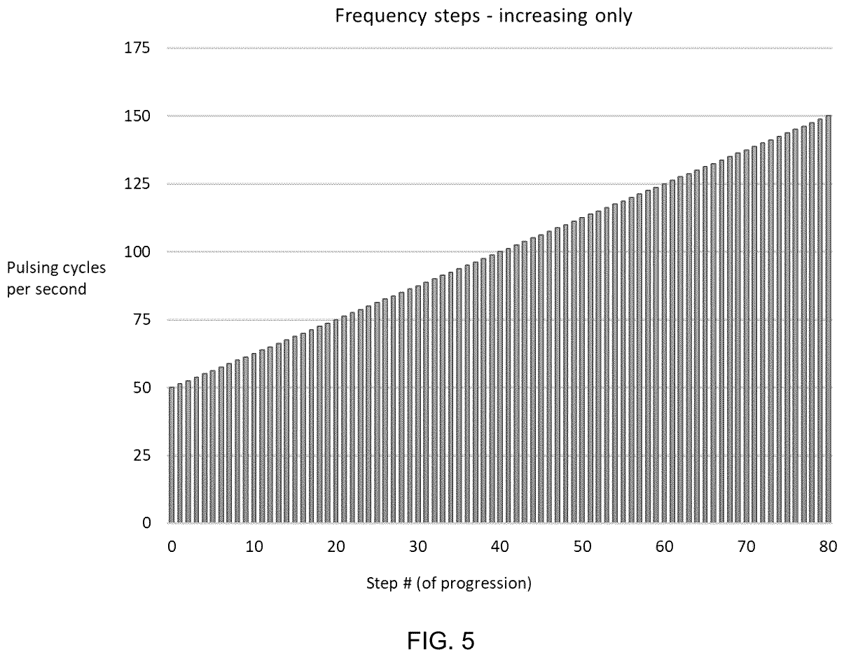

FIG. 5 illustrates a pulsing-cycle sequence comprising only a progression of pulsing cycles monotonically increasing in frequency from a minimum frequency to a maximum frequency. In the non-limiting example of FIG. 5, a schematic graph of frequency vs. step number, the progression begins at a minimum frequency of 50 pulsing cycles per second and grows to 150 pulsing cycles per second in 80 steps (discrete frequency increases). In examples, a progression can include least 20 steps and at most 200 steps, or at least 40 steps and at most 150 steps, or at least 60 steps and at most 100 steps. The minimum frequency of a progression can be at least 30 pulsing cycles per second and at most 60 pulsing cycles per second, while the maximum frequency can be is at least 100 pulsing cycles per second and at most 200.

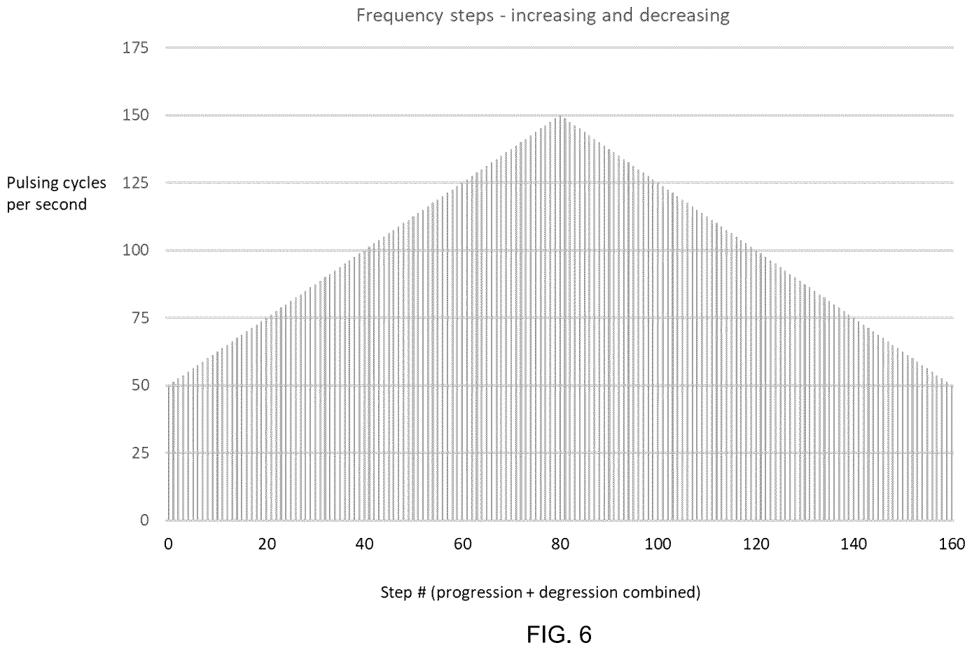

FIG. 6 illustrates a pulsing-cycle sequence comprising a progression of pulsing cycles monotonically increasing in frequency from a minimum frequency to a maximum frequency, followed by a degression of pulsing cycles monotonically decreasing in frequency from approximately the same maximum frequency to approximately the same minimum frequency. In the non-limiting example of FIG. 6, a schematic graph of frequency vs. step number, the progression begins at a minimum frequency of 50 pulsing cycles per second and grows to 150 pulsing cycles per second in 80 steps (discrete frequency increases). the degression begins at the maximum frequency of 150 pulsing cycles per second and decreases to 50 pulsing cycles per second in 80 steps (discrete frequency decreases). In other examples, not shown the degression can be of longer or shorter duration than the progression immediately preceding it, and/or can include a larger or smaller number of discrete steps.

In embodiments, the pulsing sequences repeat without change for the duration of a treatment session in the absence of a user input changing one or more of the parameters of the sequences. The duration of the treatment session can be set in advance or can be shortened or extended by a user at any time.

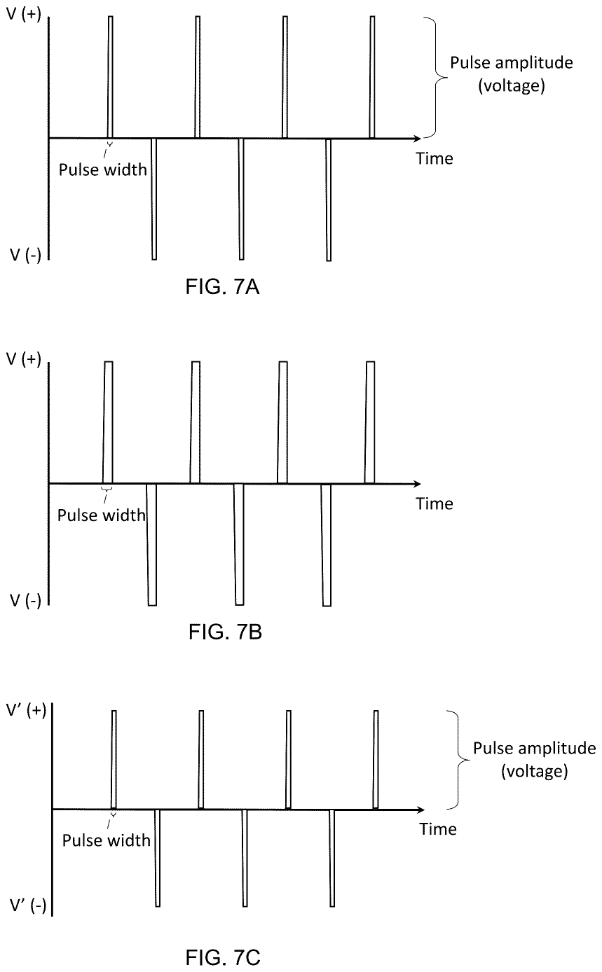

A user of an apparatus 100 for reliving symptoms of endometriosis can have access to a user interface 109 for adjusting any one of a number of treatment parameters. Any of the adjustments may be effective to increase comfort or decrease discomfort, and/or to increase the perceived efficacy of the treatment. For example, any of the adjustments may be made in response to a state of uterine contractions, e.g., too few or too many, too weak or too strong, and/or too frequent or too infrequent. Additionally or alternatively, any of the adjustments may be made in response to a state of a pain of secondary dysmenorrhea. The following paragraphs, with reference to FIGS. 7A-C, 8A-B, 9A-B, 10A-C and 11A-C describe user adjustments to individual parameters. In some embodiments, any number of parameters can be user-adjusted, whether in tandem or in sequence, to achieve desired results. All of the user adjustments shown or described are for purposes of illustration and are not in any way limiting as to the extent of possible user adjustments.

We now refer to FIGS. 7A, 7B and 7C, which show a succession of schematic graphs of voltage vs. time illustrating user adjustments of pulse width (duration) and or of pulse amplitude (intensity). FIG. 7A represents an initial treatment pattern, with pulse amplitude, i.e., maximum voltage per pulse, and pulse width, i.e., pulse duration, as per the example of FIG. 4B. FIG. 7B illustrates a hypothetical adjustment, e.g., a user-adjustment, to pulse width. In an example, the pulse width is changed by a user input received through the user interface 109 of an apparatus 100, e.g., through input mechanism 108_{PUW} shown in FIG. 2, to double the pulse width from 100 microseconds (in FIG. 7A) to 200 microseconds. FIG. 7C illustrates a hypothetical adjustment, e.g., a user-adjustment, to pulse amplitude, which is the maximum absolute value of pulse voltage. Typical maximum voltage is in the range 60V to 80V. In the example of FIG. 7C, the pulse amplitude is changed by a user input received through the user interface 109 of an apparatus 100, e.g., through voltage-adjustment input mechanism 108_{VA} shown in FIG. 2, to reduce the maximum voltage of the pulses from 80V (in FIG. 7A) to 60V.

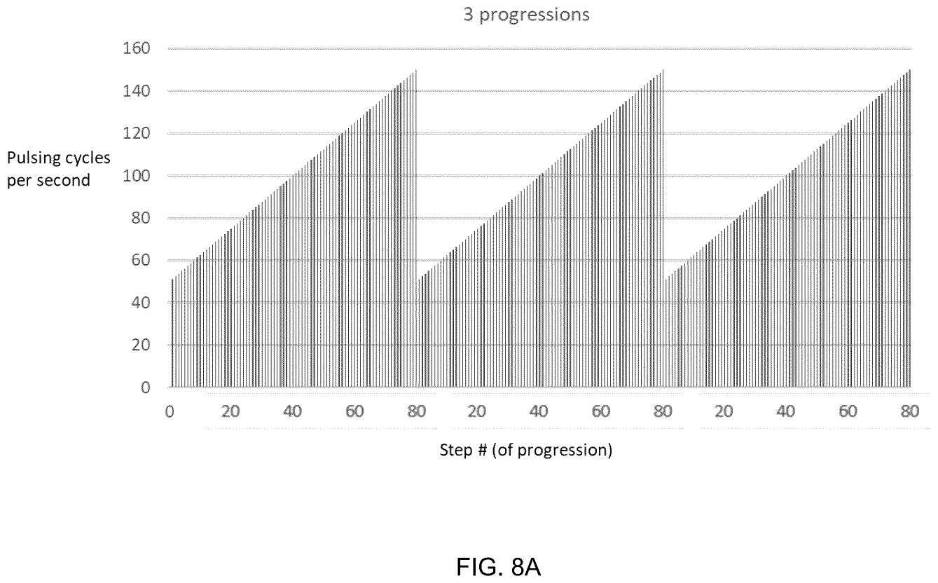

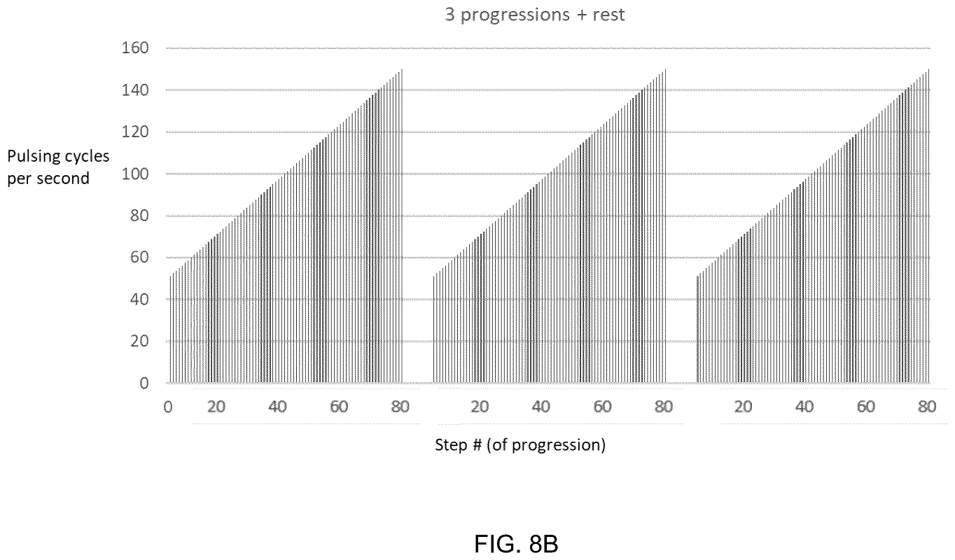

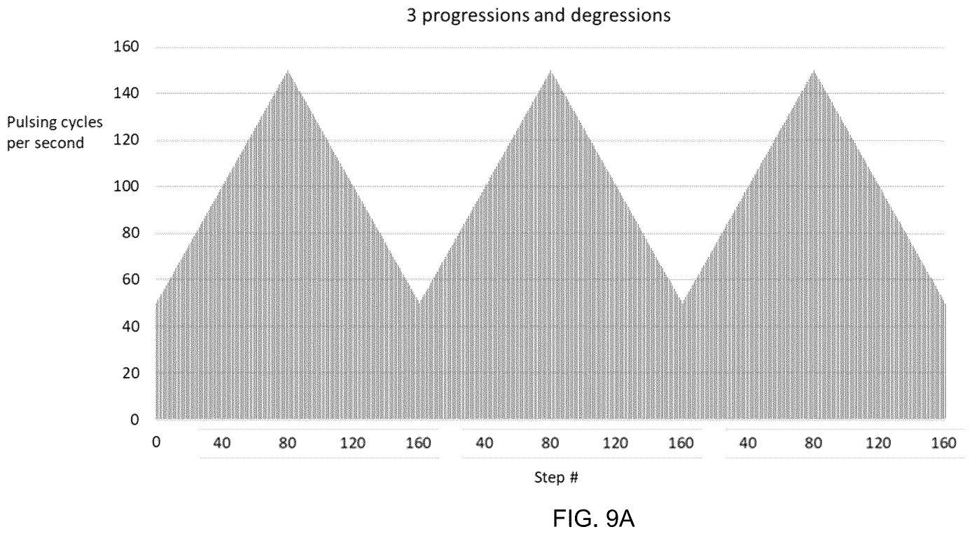

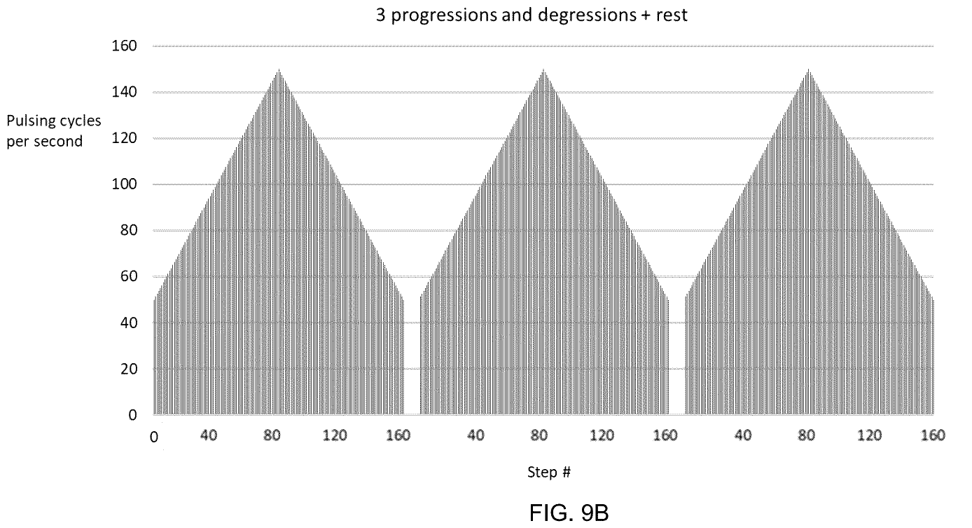

We now refer to FIGS. 8A-B and 9A-B. FIG. 8A shows a schematic graph of frequency vs. step number in which the exemplary pulsing-cycle sequence of FIG. 5, comprising only a progression of pulsing cycles monotonically increasing in frequency, is repeated. FIG. 8B illustrates a hypothetical adjustment, e.g., a user-adjustment, to between-sequence rest periods. Specifically, FIG. 8B shows a user-adjusted rest period in contrast to FIG. 8A, which shows the pulsing sequences repeating without intervening rest periods. In the example of FIG. 8B, a rest period has been added by a user input received through the user interface 109, e.g., through the between-sequence rest input mechanism 108_{BSR} shown in FIG. 2. FIG. 9A shows a schematic graph of frequency vs. step number in which the exemplary pulsing-cycle sequence of FIG. 6, comprising a progression of pulsing cycles monotonically increasing in frequency followed by a corresponding degression of pulsing cycles monotonically decreasing in frequency, is repeated. FIG. 9B illustrates a hypothetical adjustment, e.g., a user-adjustment, to between-sequence rest periods. Specifically, FIG. 9B shows a user-adjusted rest period in contrast to FIG. 9A, which shows the sequences repeating without intervening rest periods. In the example of FIG. 9B, a rest period has been added by a user input received through the user interface 109, e.g., through the between-sequence rest input mechanism 108_{BSR} shown in FIG. 2.

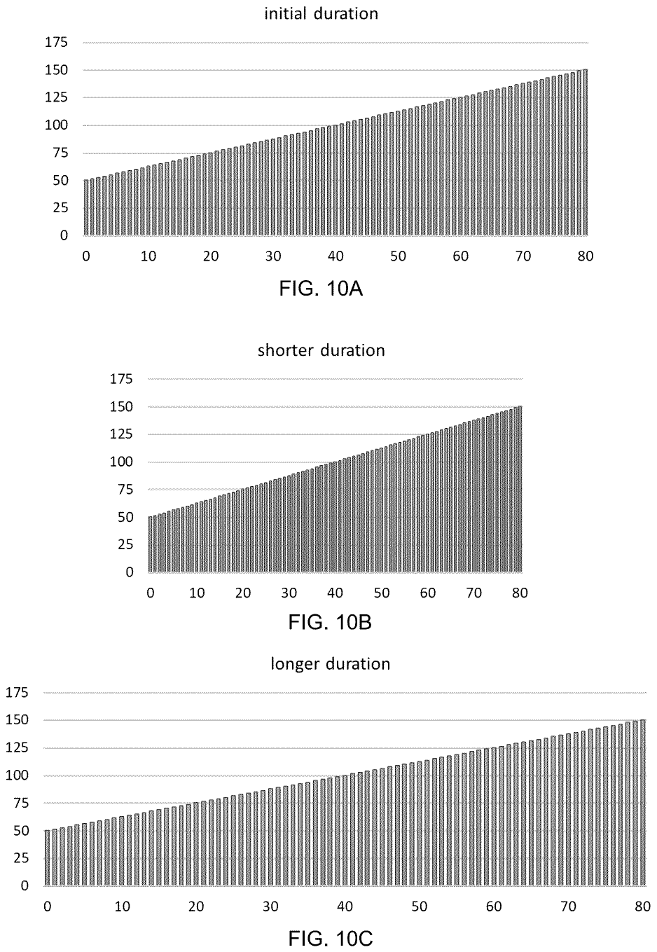

We now refer to FIGS. 10A-C and 11A-C.

In embodiments, a user can be provided with either or both of two options for changing a parameter of a progression (and, by extension, of a corresponding degression following a progression). FIGS. 10A-C are schematic graphs of frequency vs. step number, and relate to changing the duration of a progression of pulsing cycles (regardless of whether the number of steps per progression is adjusted). In some embodiments, the minimum duration of a progression is 3 seconds, and the maximum duration is 20 seconds, or 19 seconds, or 18 seconds, or 17 seconds, or 16 seconds, or 15 seconds, or 14 seconds, or 13 seconds, or 12 seconds. ‘Minimum duration’ of a progression refers to the minimum length of time believed to be effective in relieving one or more symptoms of endometriosis. In some embodiments, the minimum duration of a progression is 4 seconds, and the maximum duration is 20 seconds, or 19 seconds, or 18 seconds, or 17 seconds, or 16 seconds, or 15 seconds, or 14 seconds, or 13 seconds, or 12 seconds. According to some embodiments, the minimum duration of a progression is 5 seconds, and the maximum duration is 20 seconds, or 19 seconds, or 18 seconds, or 17 seconds, or 16 seconds, or 15 seconds, or 14 seconds, or 13 seconds, or 12 seconds. According to some embodiments, the minimum duration of a progression is 6 seconds, and the maximum duration is 20 seconds, or 19 seconds, or 18 seconds, or 17 seconds, or 16 seconds, or 15 seconds, or 14 seconds, or 13 seconds, or 12 seconds. FIG. 10A, as in FIG. 5, shows a progression from a minimum frequency of 50 pulsing cycles per second to a maximum frequency of 150 pulsing cycles per second in 80 steps. FIG. 10B shows a reduction in the duration, in seconds, of the progression of FIG. 10A, and FIG. 10C shows an increase in the duration, in seconds, of the progression of FIG. 10A. In an illustrative and non-limiting example, the initial duration of the progression of FIG. 10A is 10 seconds, the shortened duration shown in FIG. 10B is 6 seconds, and the extended duration shown in FIG. 10C is 13 seconds. In the examples of FIGS. 10B and 10C, the progression duration has been adjusted by a user input received through the user interface 109, e.g., through the progression-duration adjustment input mechanism 108_{PD} shown in FIG. 2.

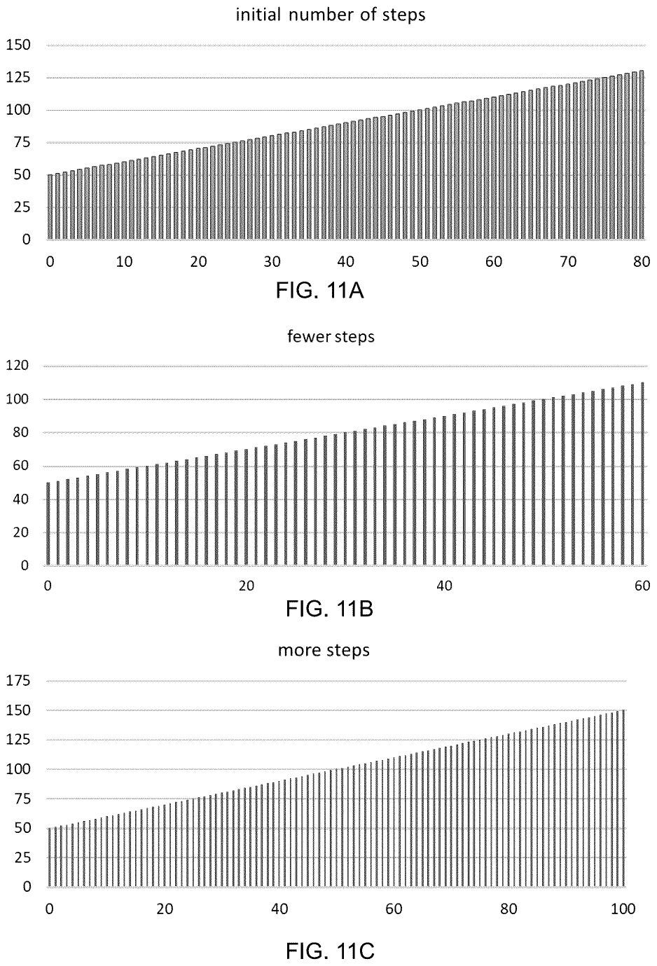

FIGS. 11A-C are schematic graphs of frequency vs. step number, and relate to changing the number of steps per progression (regardless of whether the progression duration is adjusted). According to some embodiments, the monotonic increase of a progression is in steps of at least 1 pulsing cycle per second, and limited to at most 10 pulsing cycles per second. In some embodiments, the monotonic increase of an entire progression requires at least 20 discrete steps, or at least 30 discrete steps, or at least 40 discrete steps, or at least 50 discrete steps, or at least 60 discrete steps, and at most 200 steps. The minimum number of steps in a progression refers to the minimum number of steps believed to be effective in relieving one or more symptoms of endometriosis. In some embodiments, the monotonic increase of an entire progression requires at least 20 discrete steps, or at least 30 discrete steps, or at least 40 discrete steps, or at least 50 discrete steps, or at least 60 discrete steps, and at most 150 steps. In some embodiments, the monotonic increase of an entire progression requires at least 20 discrete steps, or at least 30 discrete steps, or at least 40 discrete steps, or at least 50 discrete steps, or at least 60 discrete steps, and at most 100 steps. FIG. 11A, as in FIG. 5, shows a progression from a minimum frequency of 50 pulsing cycles per second to a maximum frequency of 150 pulsing cycles per second in 80 steps. FIG. 11B shows a reduction in the number of steps (relative to FIG. 11A) to 60, and FIG. 11C shows an increase in the number of steps (relative to FIG. 11A) to 100 steps. In the examples of FIGS. 11B and 11C, the number of steps has been adjusted by a user input received through the user interface 109, e.g., through the progression-steps adjustment input mechanism 108_{PS} shown in FIG. 2.

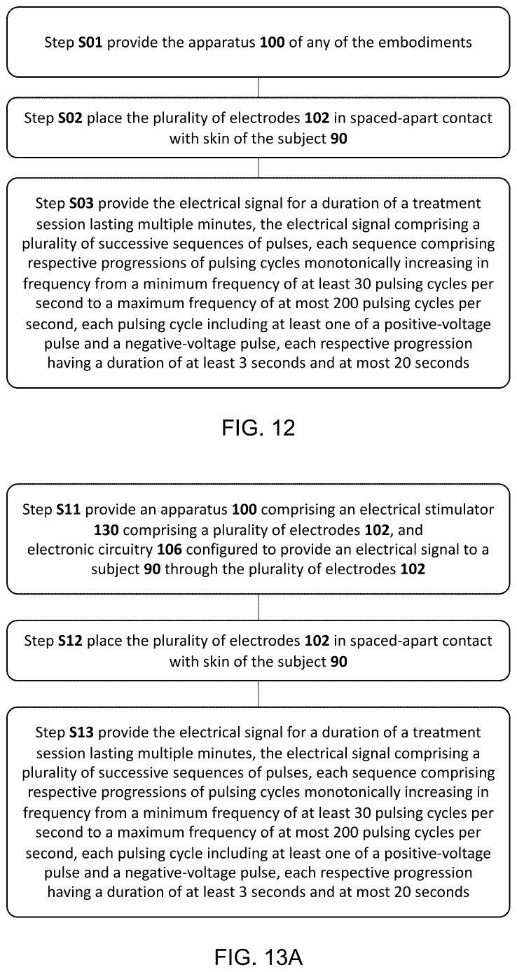

Referring now to FIG. 12, a method is disclosed for relieving one of more symptoms of endometriosis. As illustrated by the flowchart in FIG. 12, the method comprises at least the three method steps S01, S02 and S03:
Step S01 includes providing an apparatus 100 according to any of the embodiments disclosed herein.
Step S02 includes placing the plurality of electrodes 102 in spaced-apart contact with skin of the subject 90.
Step S03 includes providing an electrical signal for a duration of a treatment session lasting multiple minutes. Said electrical signal comprises a plurality of successive sequences of pulses, wherein each sequence comprises respective progressions of pulsing cycles monotonically increasing in frequency from a minimum frequency of at least 30 pulsing cycles per second to a maximum frequency of at most 200 pulsing cycles per second. Each pulsing cycle includes at least one of: a positive-voltage pulse and a negative-voltage pulse. Each respective progression has a duration of at least 3 seconds and at most 20 seconds. In some embodiments, carrying out Step S03 is effective, during the treatment session, (i) to relieve a pain of secondary dysmenorrhea and (ii) to promote, increase an amplitude of, and/or increase a frequency of, uterine contractions.

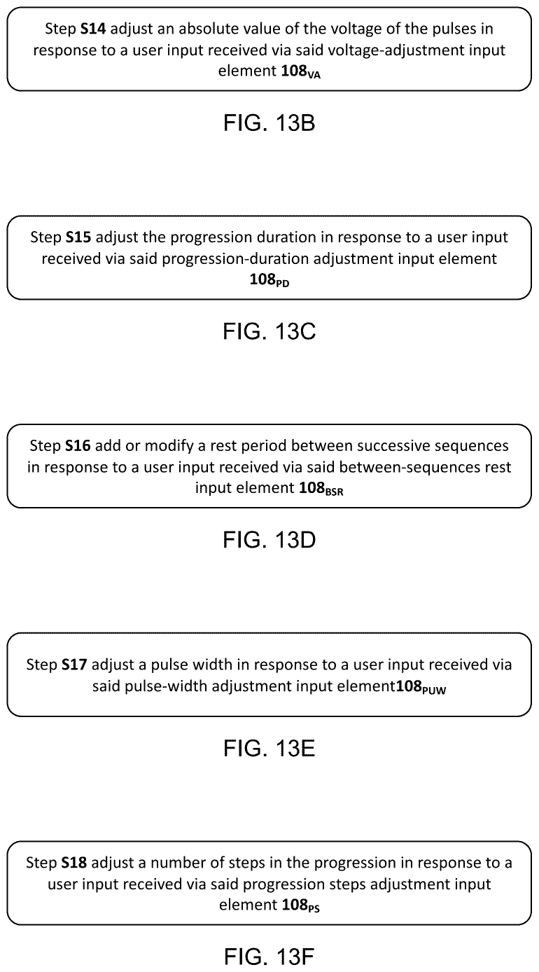

Referring now to FIG. 13A, a method is disclosed for relieving one of more symptoms of endometriosis. As illustrated by the flowchart in FIG. 12, the method comprises at least the three method steps S11, S12 and S13:
Step S11 includes providing an apparatus 100 comprising (i) an electrical stimulator 130 comprising a plurality of electrodes 102, and (ii) electronic circuitry 106 configured to provide an electrical signal to a subject 90 through the plurality of electrodes 102.
Step S12 includes placing the plurality of electrodes 102 in spaced-apart contact with skin of the subject 90.
Step S13 includes provide an electrical signal for a duration of a treatment session lasting multiple minutes. Said electrical signal comprises a plurality of successive sequences of pulses, wherein each sequence comprises respective progressions of pulsing cycles monotonically increasing in frequency from a minimum frequency of at least 30 pulsing cycles per second to a maximum frequency of at most 200 pulsing cycles per second. Each pulsing cycle includes at least one of: a positive-voltage pulse and a negative-voltage pulse. Each respective progression has a duration of at least 3 seconds and at most 20 seconds. In some embodiments, each pulsing cycle includes exactly one positive-voltage pulse and exactly one negative-voltage pulse. In some embodiments, each sequence additionally comprises, following said progression of pulsing cycles monotonically increasing in frequency, a degression of pulsing cycles monotonically decreasing in frequency from a frequency that is at most 15% lower than the maximum frequency to a frequency that is at most 15% higher than the minimum frequency, each degression having a duration of at least 3 seconds and at most 20 seconds. In some embodiments, the duration of each degression is within ±15% of the duration of the respective preceding progression.

In some embodiments, Step S13 is effective to promote uterine contractions during the treatment session, and/or increase an amplitude of one or more uterine contractions during the treatment session, and/or increase a frequency of successive uterine contractions during the treatment session. In some embodiments, Step S13 is effective to relieve a pain of secondary dysmenorrhea during the treatment session.

In some embodiments, apparatus 100 additionally comprises a voltage-adjustment input element 108_{VA}, and the method additionally comprises Step S14, as illustrated by the flow chart of FIG. 13B.
Step S14 includes adjusting an absolute value of the voltage of the pulses in response to a user input received via said voltage-adjustment input element 108_{VA}.

In some embodiments, apparatus 100 additionally comprises a progression-duration adjustment input element 108_{PD}, and the method additionally comprises Step S15, as illustrated by the flow chart of FIG. 13C.
Step S15 includes adjusting the progression duration in response to a user input received via said progression-duration adjustment input element 108_{PD}.

In some embodiments, apparatus 100 additionally comprises a between-sequences rest input element 108_{BSR}, and the method additionally comprises Step S16, as illustrated by the flow chart of FIG. 13D.
Step S16 includes adding or modifying a rest period between successive sequences in response to a user input received via said between-sequences rest input element 108_{BSR}.

In some embodiments, apparatus 100 additionally comprises a pulse-width input element 108_{PUW}, and the method additionally comprises Step S17, as illustrated by the flow chart of FIG. 13E.
Step S17 includes adjusting a pulse width in response to a user input received via said pulse-width adjustment input element 108_{PUW}.

In some embodiments, apparatus 100 additionally comprises a progression-steps adjustment input element 108_{PS}, and the method additionally comprises Step S18, as illustrated by the flow chart of FIG. 13F.
Step S18 includes adjusting a number of steps in the progression in response to a user input received via said progression steps adjustment input element 108_{PS}.

Following is a non-exhaustive and non-limiting list of Inventive Concepts according to some embodiments of the disclosure. Embodiments of the invention can include any one or more of the Inventive Concepts in any combination.

Inventive Concept 1: An apparatus for relieving symptoms of endometriosis, the apparatus comprising: (a) an electrical stimulator comprising a plurality of electrodes configured to be placed in spaced-apart contact with skin of a subject; and (b) electronic circuitry configured to provide, when activated, an electrical signal for application to the subject through the plurality of electrodes, the electrical signal comprising a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second.

Inventive Concept 2: The apparatus of Inventive Concept 1, wherein each respective progression has a duration of at least 3 seconds and at most 20 seconds.

Inventive Concept 3: The apparatus of either one of Inventive Concepts 1 or 2, wherein the electronic circuitry is programmed or programmable to provide the electrical signal for a duration of a treatment session lasting multiple minutes.

Inventive Concept 4: The apparatus of any one of the preceding Inventive Concepts, wherein the electronic circuitry is programmed or programmable to provide the electrical signal for a duration of at least 10, or at least 20, or at least 50 successive sequences.

Inventive Concept 4A: The apparatus of any one of the preceding Inventive Concepts, wherein each pulsing cycle includes at least one of: a positive-voltage pulse and a negative-voltage pulse.

Inventive Concept 5: The apparatus of any one of the preceding Inventive Concepts, wherein each sequence additionally comprises, following said progression of pulsing cycles monotonically increasing in frequency, a degression of pulsing cycles monotonically decreasing in frequency from a frequency that is at most 15% lower than the maximum frequency to a frequency that is at most 15% higher than the minimum frequency, each degression having a duration of at least 3 seconds and at most 20 seconds.

Inventive Concept 6: The apparatus of Inventive Concept 5, wherein the duration of each degression is within ±15% of the duration of the respective preceding progression.

Inventive Concept 7: The apparatus of any one of the preceding Inventive Concepts, wherein the duration of each respective progression is at least 4 seconds.

Inventive Concept 8: The apparatus of any one of the preceding Inventive Concepts, wherein the duration of each respective progression is at most 15 seconds.

Inventive Concept 9: The apparatus of any one of the preceding Inventive Concepts, wherein the duration of each respective progression is at most 13 seconds.

Inventive Concept 10: The apparatus of any one of the preceding Inventive Concepts, wherein the monotonic increase is in steps of at least 1 pulsing cycle per second and at most 10 pulsing cycles per second.

Inventive Concept 11: The apparatus of any one of the preceding Inventive Concepts, wherein the monotonic increase is in at least 20 steps and at most 200 steps.

Inventive Concept 12: The apparatus of any one of the preceding Inventive Concepts, wherein the monotonic increase is in at least 40 steps and in at most 150 steps.

Inventive Concept 13: The apparatus of any one of the preceding Inventive Concepts, wherein the monotonic increase is in at least 60 steps and in at most 100 steps.

Inventive Concept 14: The apparatus of any one of the preceding Inventive Concepts, wherein a maximum absolute value of a voltage of the pulses is 80V.

Inventive Concept 15: The apparatus of any one of the preceding Inventive Concepts, wherein a maximum absolute value of a voltage of the pulses is 60V.

Inventive Concept 16: The apparatus of any one of the preceding Inventive Concepts, wherein the minimum frequency is at least 30 pulsing cycles per second and at most 60 pulsing cycles per second.

Inventive Concept 17: The apparatus of any one of the preceding Inventive Concepts, wherein the maximum frequency is at least 100 pulsing cycles per second and at most 200 pulsing cycles per second.

Inventive Concept 18: The apparatus of any one of the preceding Inventive Concepts, wherein the plurality of successive sequences in a treatment session are identical in an absence of a user input.

Inventive Concept 19: The apparatus of any one of the preceding Inventive Concepts, additionally comprising a voltage-adjustment input element, wherein the electronic circuitry is configured to adjust an absolute value of the voltage of the pulses in response to a user input received via said voltage-adjustment input element.

Inventive Concept 20: The apparatus of any one of the preceding Inventive Concepts, additionally comprising a progression-duration adjustment input element, wherein the electronic circuitry is configured to adjust the progression duration in response to a user input received via said progression-duration adjustment input element.

Inventive Concept 21: The apparatus of any one of the preceding Inventive Concepts, additionally comprising a between-sequences rest input element, wherein the electronic circuitry is configured to add or modify a rest period between successive sequences in response to a user input received via said between-sequences rest input element.

Inventive Concept 22: The apparatus of any one of the preceding Inventive Concepts, additionally comprising a pulse-width input element, wherein the electronic circuitry is configured to adjust a pulse width in response to a user input received via said pulse-width adjustment input element.

Inventive Concept 23: The apparatus of any one of the preceding Inventive Concepts, wherein, when (i) the plurality of electrodes is placed in spaced-apart contact with skin of the subject, and (ii) the electronic circuitry is activated to provide said electrical signal such that said electrical signal is applied to the subject through the plurality of electrodes, the electrical signal is effective, during the treatment session, to (i) relieve a pain of secondary dysmenorrhea and/or (ii) to promote, increase an amplitude of, and/or increase a frequency of, uterine contractions.

Inventive Concept 24: A method of relieving one or more symptoms of endometriosis, the method comprising: (a) providing the apparatus of any one of the preceding Inventive Concepts; (b) placing the plurality of electrodes in spaced-apart contact with skin of the subject; and (c) providing the electrical signal for a duration of a treatment session lasting multiple minutes, wherein the electrical signal comprises a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of a positive-voltage pulse and a negative-voltage pulse, wherein each respective progression has a duration of at least 3 seconds and at most 20 seconds.

Inventive Concept 25: The method of Inventive Concept 23, wherein the providing of the electrical signal is effective, during the treatment session, (i) to relieve a pain of secondary dysmenorrhea and (ii) to promote, increase an amplitude of, and/or increase a frequency of, uterine contractions.

Inventive Concept 26: A method of relieving one or more symptoms of endometriosis, the method comprising: (a) providing an apparatus comprising: (i) an electrical stimulator comprising a plurality of electrodes, and (ii) electronic circuitry configured to provide an electrical signal to a subject through the plurality of electrodes; (b) placing the plurality of electrodes in spaced-apart contact with skin of the subject; and (c) providing the electrical signal for a duration of a treatment session lasting multiple minutes, wherein the electrical signal comprises a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of a positive-voltage pulse and a negative-voltage pulse.

Inventive Concept 27: The method of Inventive Concept 26, wherein providing the electrical signal includes providing the electrical signal for a duration of at least 10, or at least 20, or at least 50 successive sequences.

Inventive Concept 28: The method of Inventive Concept 27, wherein each respective progression has a duration of at least 3 seconds and at most 20 seconds.

Inventive Concept 29: The method of either one of Inventive Concepts 27 or 28, wherein each sequence additionally comprises, following said progression of pulsing cycles monotonically increasing in frequency, a degression of pulsing cycles monotonically decreasing in frequency from a frequency that is at most 15% lower than the maximum frequency to a frequency that is at most 15% higher than the minimum frequency, each degression having a duration of at least 3 seconds and at most 20 seconds.

Inventive Concept 30: The method of Inventive Concept 29, wherein the duration of each degression is within ±15% of the duration of the respective preceding progression.

Inventive Concept 31: The method of any one of Inventive Concepts 26 to 30, wherein the providing of the electrical signal is effective to promote uterine contractions during the treatment session.

Inventive Concept 32: The method of any one of Inventive Concepts 26 to 30, wherein the providing of the electrical signal is effective to increase an amplitude of one or more uterine contractions during the treatment session.

Inventive Concept 33: The method of any one of Inventive Concepts 26 to 30, wherein the providing of the electrical signal is effective to increase a frequency of successive uterine contractions during the treatment session.

Inventive Concept 34: The method of any one of Inventive Concepts 26 to 30, wherein the providing of the electrical signal is effective to relieve a pain of secondary dysmenorrhea during the treatment session.

Inventive Concept 35: The method of any one of Inventive Concepts 30 to 33, wherein the providing of the electrical signal is also effective to relieve a pain of secondary dysmenorrhea during the treatment session.

Inventive Concept 36: The method of any one of Inventive Concepts 26 to 35, wherein the duration of each respective progression is at least 4 seconds.

Inventive Concept 37: The method of any one of Inventive Concepts 26 to 36, wherein the duration of each respective progression is at most 15 seconds.

Inventive Concept 38: The method of any one of Inventive Concepts 26 to 36, wherein the duration of each respective progression is at most 13 seconds.

Inventive Concept 39: The method of any one of Inventive Concepts 26 to 38, wherein the monotonic increase is in steps of at least 1 pulsing cycle per second and at most 10 pulsing cycles per second.

Inventive Concept 40: The method of any one of Inventive Concepts 26 to 39, wherein the monotonic increase is in at least 20 steps and at most 200 steps.

Inventive Concept 41: The method of any one of Inventive Concepts 26 to 39, wherein the monotonic increase is in at least 40 steps and at most 150 steps.

Inventive Concept 42: The method of any one of Inventive Concepts 25 to 39, wherein the monotonic increase is in at least 60 steps and at most 100 steps.

Inventive Concept 43: The method of any one of Inventive Concepts 26 to 42, wherein a maximum absolute value of a voltage of the pulses is 80V.

Inventive Concept 44: The method of any one of Inventive Concepts 26 to 42, wherein a maximum absolute value of a voltage of the pulses is 60V.

Inventive Concept 45: The method of any one of Inventive Concepts 26 to 44, wherein the minimum frequency is at least 30 pulsing cycles per second and at most 60 pulsing cycles per second.

Inventive Concept 46: The method of any one of Inventive Concepts 26 to 45, wherein the maximum frequency is at least 100 pulsing cycles per second and at most 200 pulsing cycles per second.

Inventive Concept 47: The method of any one of Inventive Concepts 26 to 46, wherein the plurality of successive sequences are identical in an absence of a user input.

Inventive Concept 48: The method of any one of Inventive Concepts 26 to 47, wherein the apparatus additionally comprises a voltage-adjustment input element, and the method additionally comprises adjusting an absolute value of the voltage of the pulses in response to a user input received via said voltage-adjustment input element.

Inventive Concept 49: The method of any one Inventive Concepts 26 to 48, wherein the apparatus additionally comprises a progression-duration adjustment input element, and the method additionally comprises adjusting the progression duration in response to a user input received via said progression-duration adjustment input element.

Inventive Concept 50: The method of any one Inventive Concepts 26 to 49, wherein the apparatus additionally comprises a between-sequences rest input element, and the method additionally comprises adding or modifying a rest period between successive sequences in response to a user input received via said between-sequences rest input element.

Inventive Concept 51: The method of any one of Inventive Concepts 26 to 50, wherein the apparatus additionally comprises a pulse-width input element, and the method additionally comprises adjusting a pulse width in response to a user input received via said pulse-width adjustment input element.

Inventive Concept 52: The method of any one of Inventive Concepts 46 to 51, wherein the user input is in response to a state of uterine contractions.

Inventive Concept 53: The method of any one of Inventive Concepts 46 to 51, wherein the user input is in response to a state of a pain of secondary dysmenorrhea.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

1. An apparatus for relieving symptoms of endometriosis, the apparatus comprising:
a. an electrical stimulator comprising a plurality of electrodes configured to be placed in spaced-apart contact with skin of a subject; and
b. electronic circuitry configured to provide, when activated, an electrical signal for application to the subject through the plurality of electrodes, the electrical signal comprising a plurality of successive sequences of pulses, each sequence comprising respective progressions of pulsing cycles monotonically increasing in frequency from a minimum frequency of at least 30 pulsing cycles per second to a maximum frequency of at most 200 pulsing cycles per second, the maximum frequency being at least 40 pulsing cycles per second higher than the minimum frequency, each pulsing cycle including at least one of: a positive-voltage pulse and a negative-voltage pulse.

2. The apparatus of claim 1, wherein each respective progression has a duration of at least 3 seconds and at most 20 seconds.

3. The apparatus of either one of claims 1 or 2, wherein the electronic circuitry is programmed or programmable to provide the electrical signal for a duration of a treatment session lasting at least two minutes.

4. The apparatus of any one of the preceding claims, wherein the electronic circuitry is programmed or programmable to provide the electrical signal for a duration of at least 10, or at least 20, or at least 50 successive sequences.

5. The apparatus of any one of the preceding claims, wherein each sequence additionally comprises, following said progression of pulsing cycles monotonically increasing in frequency, a degression of pulsing cycles monotonically decreasing in frequency from a frequency that is at most 15% lower than the maximum frequency to a frequency that is at most 15% higher than the minimum frequency, each degression having a duration of at least 3 seconds and at most 20 seconds.

6. The apparatus of claim 5, wherein the duration of each degression is within ±15% of the duration of the respective preceding progression.

7. The apparatus of any one of the preceding claims, wherein the duration of each respective progression is at least 4 seconds and at most 15 seconds, or at least 4 seconds and at most 13 seconds.

8. The apparatus of any one of the preceding claims, wherein the monotonic increase is in steps of at least 1 pulsing cycle per second and at most 10 pulsing cycles per second.

9. The apparatus of any one of the preceding claims, wherein the monotonic increase is in at least 20 steps and at most 200 steps, or at least 40 steps and at most 150 steps, or at least 60 steps and at most 100 steps.

10. The apparatus of any one of the preceding claims, wherein a maximum absolute value of a voltage of the pulses is no higher than 80V, or no higher than 60V.

11. The apparatus of any one of the preceding claims, wherein the minimum frequency is at least 30 pulsing cycles per second and at most 60 pulsing cycles per second, and the maximum frequency is at least 100 pulsing cycles per second and at most 200 pulsing cycles per second.

12. The apparatus of any one of the preceding claims, wherein the plurality of successive sequences in a treatment session are identical in an absence of a user input.

13. The apparatus of any one of the preceding claims, additionally comprising at least one of the following input elements:
i. a voltage-adjustment input element for adjusting an absolute value of the voltage of the pulses,
ii. a progression-duration adjustment input element, for adjusting the progression duration,
iii. a between-sequences rest input element, for adding or modifying a rest period between successive sequences, and
iv. a pulse-width input element, for adjusting a pulse width.

14. The apparatus of any one of the preceding claims, wherein, when (i) the plurality of electrodes is placed in spaced-apart contact with skin of the subject, and (ii) the electronic circuitry is activated to provide said electrical signal such that said electrical signal is applied to the subject through the plurality of electrodes, the electrical signal is effective, during the treatment session, to (i) relieve a pain of secondary dysmenorrhea and/or (ii) to promote, increase an amplitude of, and/or increase a frequency of, uterine contractions.
